(19) United States
(12) Patent Application Publication
(10) Pub. No.: US 2012/0078187 A1
(43) Pub. Date: Mar. 29, 2012

(54) FLEXIBLE INTRODUCER SHEATH

(75) Inventor: Dennis J. Delap, Bloomington, IN (US)

(73) Assignee: Cook Incorporated, Bloomington, IN (US)

(21) Appl. No.: 12/888,909

(22) Filed: Sep. 23, 2010

(51) Int. Cl. A61M 25/00; B29C 53/60

(52) U.S. Cl. 604/171; 604/526; 156/143

(57) ABSTRACT

A sheath with improved kink resistance and/or rupture resistance is provided. The sheath includes an inner liner defining a passageway extending longitudinally therethrough. A coil is fitted around at least a part of the inner liner. An outer layer adapted to adhere to the inner liner between the windings of the coil is positioned longitudinally over the coil. Lateral edges can have inwardly curved and outwardly curved portions. Lateral edges can be further structured and arranged to define an asymmetric spacing therebetween. The asymmetric spacing is configured to improve at least one of kink resistance and rupture resistance of the sheath when in a bent configuration. The compressive and tensile strength of the polymer material can be utilized in a manner to inhibit coil segments from sliding along each other or overlapping one another, thereby preventing premature tearing of the material when the sheath is bent.

### TECHNICAL FIELD

This present disclosure relates to a medical apparatus suitable for accessing a target site within the body of a patient, and more particularly, to a sheath suitable for use in introducing items like therapeutic agents or an interventional device into a bodily passageway of a patient.

### BACKGROUND

Catheters are in widespread use in the medical field for delivering a medical interventional device, such as a stent, to a target site within a bodily passageway of a patient, such as the vasculature. In order to reach the target site, catheters are often required to traverse tortuous pathways having sharp bends and angles. In some instances, and particularly when traversing such tortuous pathways, catheters exhibit a tendency either to kink and/or rupture to a point of failure. Kinking in the wall of the catheter will likely occur radially along inside of the bending curvature due to compression failure of the material, and usually will occur before rupturing. Rupturing in the wall of the catheter will likely occur radially along the outside of the bending curvature due to tensile failure of the material. Kinking in particular reduces, and often collapses, the effective luminal diameter of the catheter, thereby rendering the catheter essentially unsuitable for its intended use. For instance, kinking while the catheter is in the vasculature can make advancement or withdrawal of the catheter difficult.

The tendency of a catheter to kink or rupture is increased when it is used to introduce an interventional device into one of the many smaller vessels that branch off from major vessels. In this event, the catheter may have insufficient bending flexibility at the very point where bending flexibility is most desired in order to enable proper positioning of the interventional device. Since the vessels are smaller vessels, the outer diameter of the catheter must be similarly sized in order to fit within the vessel, while having a sufficiently large luminal diameter for the interventional device. In order to optimize the relationship between the outer diameter of the catheter and its luminal diameter, it is desirable to form the wall of the catheter as thin as possible. A thin-wall catheter, however, often has difficulty tracking through narrow vessels, and may even result in an increased propensity to kink.

One particularly effective thin-walled catheter having an improved kink resistance is disclosed in U.S. Pat. No. 5,380,304 to Parker, which is incorporated by reference in its entirety. Here, the catheter comprises an inner liner formed of a lubricious inner liner. A coil is fitted around the inner liner, and an outer jacket formed of a heat-formable material surrounds the inner liner and coil. The heat-formable material is heat shrunk onto the outer surface of the inner liner by enveloping it in a heat shrink tube, and heating the entire assembly until the heat-formable material melts. As the heat-formable material melts, it flows between the spacings of the coil turns, and bonds to the inner liner. The use of the coil in this device reinforces the sheath wall, and provides enhanced kink-resistance to an otherwise thin-walled introducer sheath. In order to minimize the cross-sectional profile (i.e., the outer diameter) of the catheter, the coil is generally formed of a flattened wire. Further, the use of a braid in combination with a coil may help reduce rupturing and/or kinking in some instances. However, as the catheter outer diameter becomes smaller for smaller vessels and the wall becomes thinner, there may be simply not enough room to include a braid.

It has been found that during extreme bending of the catheter kinking is rather prevalent. One source of kinking is the eventual lateral failure, or tearing, of the material between the coil turns due to compression or pinching by the surrounding adjacent coil segments. Consequently, the compressive strength property of the material for kinking (and tensile strength property of the material for rupture) can be a factor in failure. That is, a material having a higher compressive strength property will withstand a higher compressive force before failure. Another factor is the cross-section of the coil. In some instances, the failure of the material from compression of the surrounding adjacent coil segments can be further exacerbated when the cross-section of the coil has sharp or pointed edges. As a result, pinching from sharp edges of the coil segments causes a stress concentration that leads to premature failure. That is, failure at a compressive force that is lower than expected from the actual compressive strength property of the material. The premature failure eventually enables one of the adjacent coil segments to slide past the other, resulting in a permanent deformation that alters the luminal diameter of the catheter.

One approach to reduce the propensity of the catheter to kink as a consequence of its coils sliding past one another is to increase the wall thickness of the catheter. However, any such increase in wall thickness undesirably limits the ability of the catheter to enter a narrow vessel and reduces the diameter of the lumen when compared to the lumen of an otherwise similar thin-wall catheter. In addition, a larger diameter catheter would also necessitate the use of a larger entry opening than would otherwise be required or desired. Another approach is to use highly engineered materials with higher compressive strength property within the spacings of the coil turns. However, these materials can be very expensive compared to conventional materials, making the use of such materials undesirable for controlling manufacturing costs.

Thus, what is needed is a catheter or sheath with improved kink resistance and/or rupture resistance. In addition, what is needed is a catheter or sheath that is configured to inhibit adjacent coil segments within the catheter wall from sliding past one another during extreme bending.

### BRIEF SUMMARY

Various embodiments of sheaths are described herein having an improved kink resistance and/or rupture resistance. The sheath includes a proximal end and a distal end, and a wall that defines a passageway extending about a longitudinal axis. The sheath wall can include various components, including at least one of: an inner liner, a reinforcement structure such as a coil, and an outer layer. The inner liner can define the passageway of the sheath. The coil can be fitted around at least a part of the inner liner. The coil can have a series of windings that are spaced apart longitudinally to define spacings between adjacent coil segments. The coil has a cross-section that is defined by radially inner and outer surfaces that are interconnected by first and second lateral edges. The outer layer can be positioned longitudinally over the coil to bond to the inner liner through spacings between adjacent coil segments.

In one embodiment, the sheath includes a coil having a first lateral edge with an inwardly curved portion and a second lateral edge with an outwardly curved portion. The first lateral edge may further include an outwardly curved portion that is joined to the inwardly curved portion. The second lateral edge of the coil may further include an inwardly curved portion that is joined to the outwardly curved portion. In one aspect, the radius of curvature of the inwardly curved portion of the first lateral edge can be at least as large as the radius of curvature of the outwardly curved portion of the second lateral edge. In other aspects, the radius of curvature of the inwardly curved portion of the second lateral edge can be at least as large as the radius of curvature of the outwardly curved portion of the first lateral edge.

In another embodiment, the sheath includes a coil having the first lateral edge of a coil segment and the second lateral edge of an adjacent coil segment that are structured and arranged to define an asymmetric spacing therebetween. The asymmetric spacing can be configured to improve at least one of kink resistance and rupture resistance of the sheath when in a bent configuration. The outer portion of a first coil winding can be spaced from the outer portion of a second, adjacent coil winding at a first distance, while the inner portion of the first coil winding can be spaced from the inner portion of the second coil winding at a second distance that is different from the first distance. The inwardly curved portion of the one of the first and second lateral edges of the coil winding segment and the outwardly curved portion of the other of the first and second lateral edges of the adjacent coil winding segment may be structured and arranged with outer layer material therebetween to form a ball-and-socket interface.

A method of forming a sheath of one of the embodiments described herein is also provided. The method can include one or more of the following steps: providing an inner polymer liner with a passageway extending therethrough and an outer surface; positioning the inner polymer liner around a mandrel; positioning a coil around the inner polymer liner, the coil having a series of windings being spaced apart longitudinally, a cross-section defined by radially inner and outer surfaces interconnected by first and second lateral edges, wherein the first lateral edge includes an inwardly curved portion and the second lateral edge includes an outwardly curved portion; applying an outer polymer layer over at least a portion of the coil; and exposing an assembly comprising the mandrel, inner polymer liner, coil and outer polymer layer to a sufficient amount of heat to at least partially melt the outer polymer layer such that a bond is formed between outer polymer layer and the inner polymer liner. During melting, material of the outer polymer layer can be disposed between the spacings defined by the first lateral edge of a first coil segment and the second lateral edge of a second, adjacent coil segment.

### BRIEF DESCRIPTION OF THE DRAWINGS

FIG. 1 is a side elevation view of a sheath for use with a medical device, partially in section, and with a portion broken away.

FIG. 2 is a cross-sectional view of a portion of the sheath of FIG. 1.

FIGS. 3A-3E are cross-sectional views of various coil embodiments that can be used in a sheath.

FIG. 4A is a partially cross-sectional view of a sheath during bending.

FIG. 4B is an enlarged view of a portion of the sheath in FIG. 4A, depicting the compressive stress distribution of sheath material.

FIG. 4C is a partially cross-sectional view of a sheath with an alternative coil configuration.

FIG. 5 is a perspective view of a wire before being wound as a coil for use in a sheath.

### DETAILED DESCRIPTION OF THE DRAWINGS AND THE PRESENTLY PREFERRED EMBODIMENTS

For the purposes of promoting an understanding of the principles of this disclosure, reference will now be made to the embodiments illustrated in the drawings, and specific language will be used to describe the same.

In the following discussion, the terms “proximal” and “distal” will be used to describe the opposing axial ends of the inventive sheath, as well as the axial ends of various component features. The term “proximal” is used in its conventional sense to refer to the end of the apparatus (or component thereof) that is closest to the operator during use of the apparatus. The term “distal” is used in its conventional sense to refer to the end of the apparatus (or component thereof) that is initially inserted into the patient, or that is closest to the patient during use.

Various embodiments of sheaths are described herein having an improved kink resistance and/or rupture resistance. The sheaths are configured in such a way that adjacent coil segments cooperatively interact with the wall material therebetween to better utilize the full compressive and/or tensile strength property of material between the adjacent coil segments along the respective inside and outside of the bending curvature. To this end, the material can maintain its structural integrity to inhibit adjacent coil segments from sliding past one another when the sheath is bent at an extreme radius of curvature, thereby increasing the kink resistance and/or rupture resistance of the sheath. This arrangement can allow the sheath to be bent at a tighter bending radius of curvature than previously recognized. The stress concentration may even be transferred to the material of the wall of the catheter radially along the outside of the bending curvature. One advantage of transferring the stress concentration to the outside of the bending curvature is that the integrity of the luminal wall of the sheath remains intact, thereby limiting material or coil protrusions within the lumen and allowing interventional devices or fluids to be safely removed from the lumen.

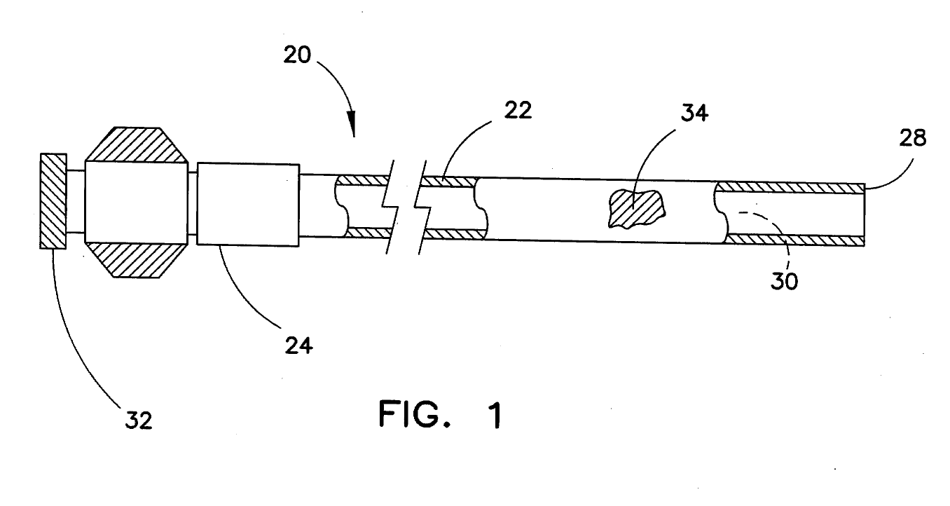

FIG. 1 shows an exemplary flexible introducer sheath 20 with an improved kink resistance and/or rupture resistance so that trackability of said sheath through tortuous pathways of the bodily passageway of the patient may be improved. Sheath 20 can be suitable for use in introducing items like therapeutic agents or an interventional device into a bodily passageway of a patient.

The sheath 20 may be useful for performing any of a variety of minimally invasive medical procedures, including, for example, angioplasty, diagnosis, chemotherapy, drainage, endoscopy, laparoscopy, and arthroscopy. Sheath 20 includes a proximal end 24, a distal end 28, and a lumen 30 extending longitudinally therethrough. Sheath 20 can extend in the distal direction from a conventional connector cap 32, as shown in FIG. 1.

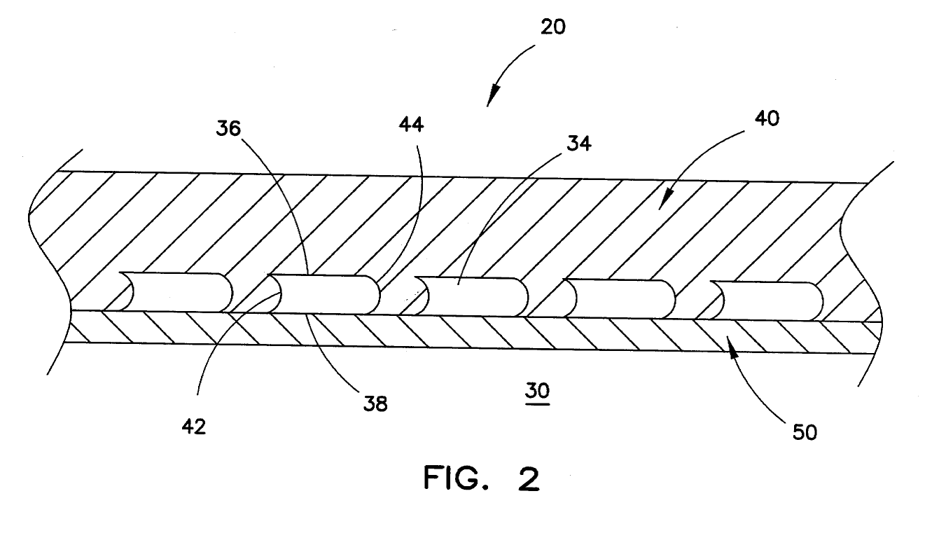

The wall 22 of the sheath. 20 can include various layers. With reference to FIG. 2, sheath 20 can include a reinforcement layer, such as a coil 34, sandwiched between an outer layer 40 and an inner liner 50. Coil 34 can be made of ribbon or wire. Additional reinforcement layers such as a braid (not shown) extending over at least part of the coil may be included in the sheath, as described in U.S. Pat. No. 6,939,337 to Parker, which is incorporated herein by reference in its entirety. When present, the braid would preferably comprise a plurality of crossed wires of circular or flat cross-section. The coil or braid can be made from a medical grade material, such as stainless steel or PEEK or other suitable polymer or metal. Other medical grade materials may also be useful for the coil and the braid, and could be the same material for each.

Outer layer 40 can be a medical grade polymer that is positioned over and contacting at least the coil in order to adhere to an outer surface 36 of one or more windings of coil 34 and/or inner liner 50 through the spacings between coil turns. Outer layer 40 can comprise heat-shrinkable (heat fused) tubing, such as a polyether block amide, polyamide (nylon), and/or polyurethane.

Inner liner 50 can be disposed beneath and along a portion of an inner surface 38 of one or more windings of coil 34. Inner liner 50 can be made of a medical grade polymer, and may have a melt temperature greater than the melt temperature of outer layer 40. Inner liner 50 can comprise a lubricious polymer, such as PTFE, although it is appreciated that other lubricious polymers as determined by those skilled in the art can be used. Inner liner 50 may be sized to define lumen 30, which is suitably sized depending on the application, e.g., the lumen can be intended for the delivery of a diagnostic or therapeutic fluid, or the removal of a fluid from the patient.

To promote adhesion between the inner and outer surfaces of the coil and the respective inner liner and outer layer, outer surface 38 and/or inner surface 36 of coil 34, as well as the outer surface of inner liner 50, can be roughened in any conventional manner, such as by machine grinding or chemical etching, to form irregularities on the surface.

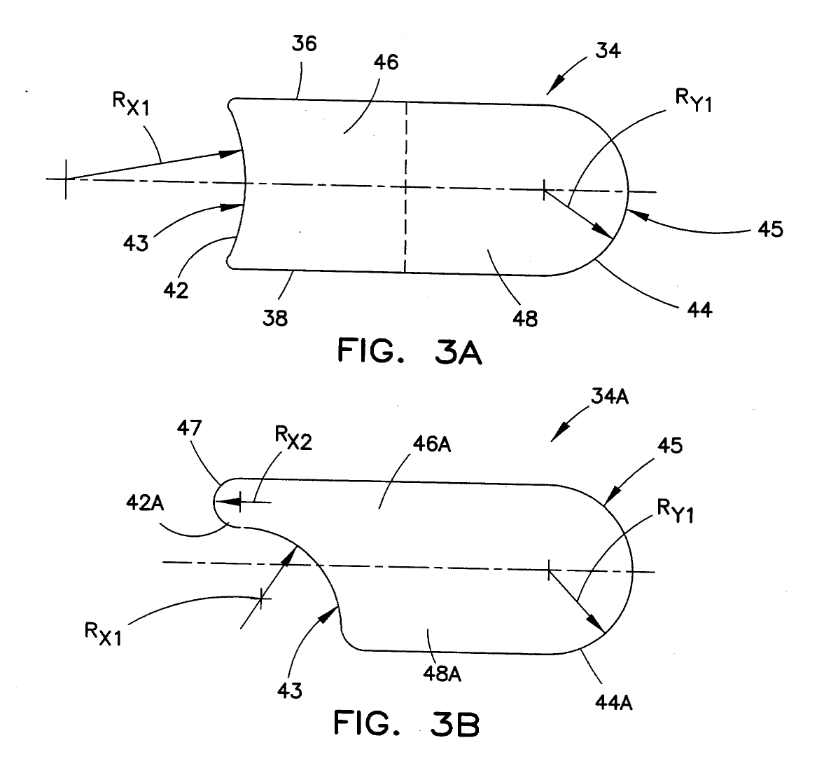

The cross-section of coil 34 can be a variety of shapes including rectangular or rounded, such as circular, oval, or semi-oval. The following figures depict preferred cross-sections of coil 34 with lateral edges 42, 44 of adjacent coil segments being structured and arranged to cooperatively interact with the outer layer material between the lateral edges so that at least one the full compressive and the full tensile strength property of the material is better utilized. The cross-section can be defined by outward surface 36 and inner surface 38 interconnected by the first lateral edge 42 and the second lateral edge 44. The cross-section can be further defined by a radial outer portion 46 and a radial inner portion 48, shown divided by a horizontal dashed line in FIG. 3A. Although the dashed line is shown through the center of the cross-section, the dashed line can be off-center such that the upper portion can be larger or smaller than the lower portion.

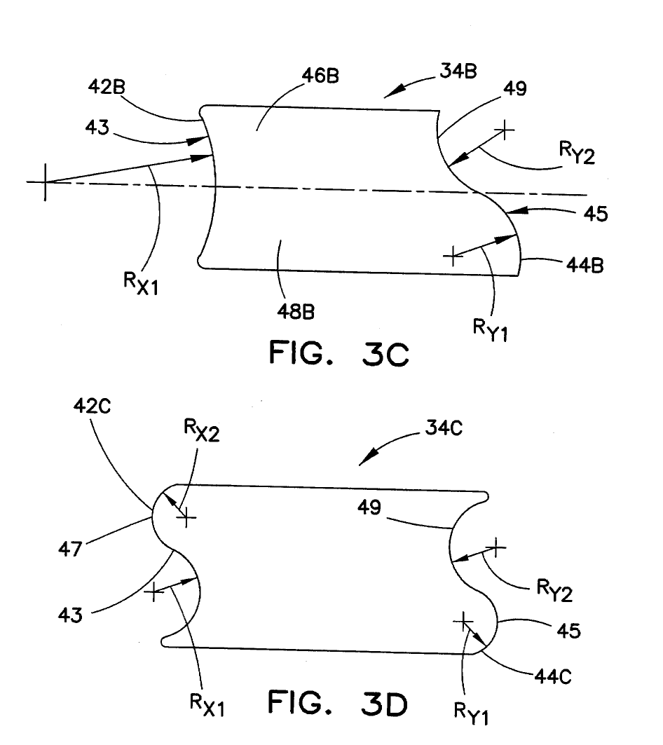

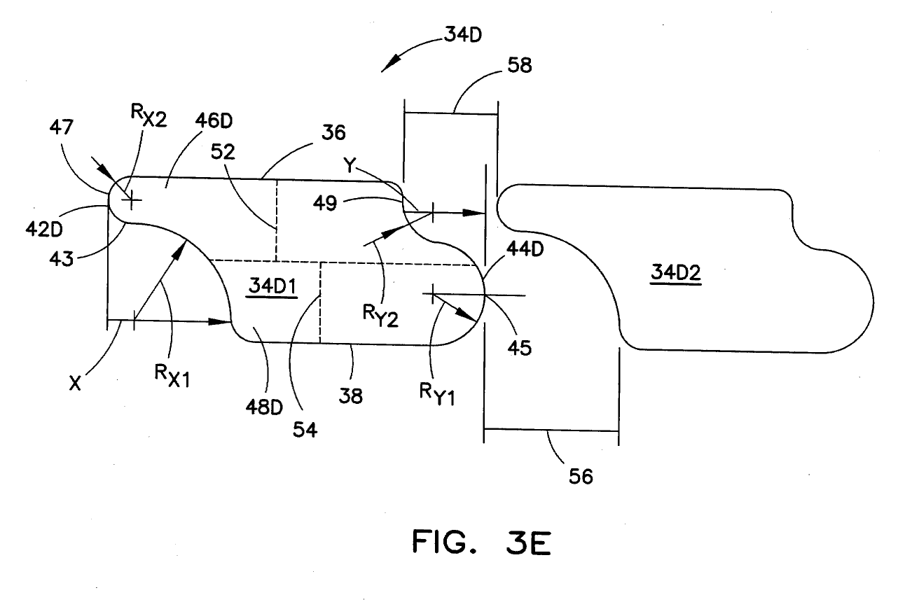

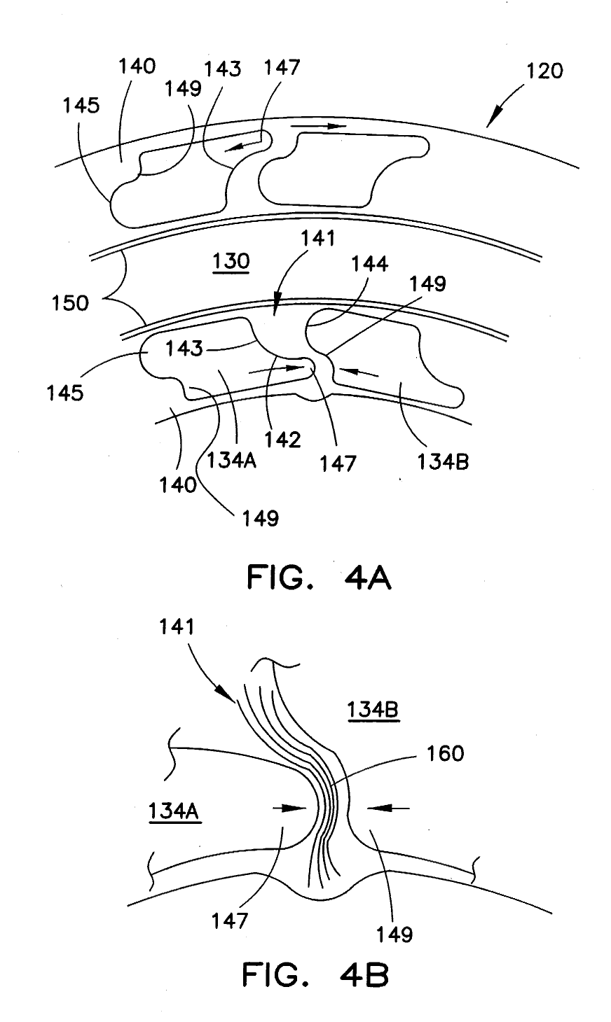

It is preferable that the shape of the lateral edges 42, 44 of the coil is configured to improve kink resistance of sheath 20. In one aspect, the lateral edges of adjacent coil segments can be shaped functionally like a ball-and-socket joint, where one lateral edge of one coil segment is shaped like the ball and the adjacent lateral edge of another coil segment is shaped like the socket. With material in the spacing between the adjacent coil segments, the adjacent coil segments can compress the material so that it can experience a more uniform compressive stress distribution, as shown for example in FIG. 4B. This arrangement can fully utilize the compressive strength of the material by inhibiting tearing of the material and inhibiting the adjacent coil segments from contacting one another. The shape of lateral edges 42, 44 may further be made smooth by grinding, sand blasting, or other process known in the art to reduce the sharpness of the cross-section of the coil. As a result, the material can maintain its integrity to mechanically obstruct adjacent coil segments from sliding past each other, thereby reducing the risk of premature tearing of the material from pinching between the adjacent coil segments.

With reference to FIG. 3A, the first lateral edge 42 of one of the embodiments of coil 34 can have an inwardly curved portion 43, and the second lateral edge 44 can have an outwardly curved portion 45. Inwardly curved portion 43 has a radius of curvature R_{X1}. Outwardly curved portion 45 has a radius of curvature R_{Y1}. Inwardly curved portion radius R_{X1} is preferably equal to or greater than outwardly curved portion radius R_{Y1}, although it can be smaller depending on the application. It would be desirable if inwardly curved portion radius R_{X1} were relatively large in order to lessen the sharpness of the edges formed by the curvature. Inwardly curved portion 43 of one coil segment is configured to function like a cupped interface surface or socket surface. Outwardly curved portion 45 of an adjacent coil segment is configured to function as a finger interface surface or ball surface.

FIG. 3B depicts another embodiment of a coil 34A that includes all of the features of coil 34 except for the following. The first lateral edge 42A of coil 34A can further include an outwardly curved portion 47 joined to inwardly curved portion 43 having the radius R_{X1}. The second lateral edge 44A includes outwardly curved portion 45 having the radius R_{Y1}. Outwardly curved portion 47 of the first lateral edge 42A has a radius of curvature R_{X2}. It can be appreciated by those skilled in the art that the first lateral edge of any of the coil embodiments can include additional outwardly curved portions and/or inwardly curved portions. Inwardly curved portion radius R_{X1} is preferably equal to or greater than outwardly curved portion radius R_{X2}. It can be further appreciated that, although outwardly curved portion 47 of the first lateral edge 42A is along the radial outer portion 46A of coil 34A, outwardly curved portion 47 can be located along the radial inner portion 48A of coil 34A.

FIG. 3C depicts another embodiment of a coil 34B that includes all of the features of coil 34 except for the following. The second lateral edge 44B of coil 34B can further include an inwardly curved portion 49 joined to outwardly curved portion 45 having the radius R_{Y1}. Inwardly curved portion 49 of the second lateral edge has a radius of curvature R_{Y2}. The first lateral edge 42B includes inwardly curved portion 43 having the radius R_{X1}. It can be appreciated by those skilled in the art that the second lateral edge of any of the coil embodiments may include additional outwardly curved portions and/or inwardly curved portions. Outwardly curved portion radius R_{Y1} is preferably equal to or greater inwardly curved portion radius R_{Y2}. It can be further appreciated that, although inwardly curved portion 49 of the second lateral edge is along the radial outer portion 46B of coil 34, inwardly curved portion 49 can be located along the radial inner portion 48B of the coil.

FIG. 3D depicts another embodiment of a coil 34C that includes all of the features of coil 34 except for the following. Coil 34C can have a series of curved portions. For instance, the first lateral edge 42C can include inwardly curved portion 43 having the radius R_{X1} joined to outwardly curved portion 47 having the radius R_{X2}. The second lateral edge 44C can include outwardly curved portion 45 having the radius R_{Y1} joined to inwardly curved portion 49 having the radius R_{Y2}. This arrangement can facilitate the inwardly curved portions 43, 49 to better cooperatively interact with the respective outwardly curved portions 45, 47 of the adjacent coil segments and the material therebetween.

As can be seen in FIGS. 3B-3C, for example, at least one of the radial outer portion and the radial inner portion of the coil can laterally extend past the respective radial inner and outer portions along different lateral edges. This arrangement can vary the lateral spacing between adjacent coil segments, thereby varying the amount of material received by the spacing. For example, in FIG. 3B the radial outer portion 46A of coil 34A extends beyond the radial inner portion 48A along the first lateral edge 42A. The extended radial outer portion 46A is shown to define outwardly curved portion 47 that has the radius R_{X2}. In another example, in FIG. 3C the radial inner portion 48B extends past the radial outer portion 46B along the second lateral edge 44. The extended radial inner portion 48B is shown to define outwardly curved portion 45 that has the radius R_{Y1}.

FIG. 3E depicts another embodiment of a coil 34D that includes various features of coils 34, 34A, 34B, 34C except for the following. The extended portions of coil 34D can offset a geometric center of the radial outer portion from a geometric center of the radial inner portion. The embodiment of coil 34D is depicted with two coil windings 34D1, 34D2 having identical cross-sections, adjacent to one another and with asymmetrical lateral edges.

The first lateral edge 42D can include inwardly curved portion 43 having the radius R_{X1} joined to outwardly curved portion 47 having the radius R_{X2}. The second lateral edge 44D can include outwardly curved portion 45 having the radius R_{Y1} joined to inwardly curved portion 49 having the radius R_{Y2}. The radial outer portion 46D of coil 34D can extend past the radial inner portion 48D along the first lateral edge 42D by a distance X, where the geometric center 52 of the radial outer portion 46D is shown by the vertical dashed line. The radial inner portion 48D can extend past the radial outer portion 46D along the second lateral edge 44D by a distance Y, where the geometric center 54 of the radial inner portion 48D is shown by the vertical dashed line.

Preferably, the distance X of extension is greater than the distance Y of extension, so that the overall distance 56 between radial inner portions of adjacent coil segments 34D1, 34D2 is greater than the overall distance 58 between radial outer portions of adjacent coil segments. In one non-limiting example, for a nominal coil width of 0.3 mm, the distance 56 can be 0.4 mm and the distance 58 can be 0.2 mm, although it can be appreciated by one skilled in the art that these dimensions can vary depending on the desired functionality of the sheath. One benefit in having a different distance between the radial inner portions of adjacent coil segments in comparison to the distance between the radial outer portions of adjacent coil segments is that there is additional material in the spacing between adjacent coil segments to be stressed, thereby lowering the corresponding percent strain of the material. This arrangement can further facilitate the utilization of the full compressive and/or tensile strength of the structure in the final assembly in a manner that results in improved the kink resistance and/or rupture resistance of the sheath. Another benefit of this arrangement is the further inhibition of coil sliding or overlapping one another.

FIG. 4A depicts a sheath 120, that is substantially identical to any of the embodiments of sheath 20 described herein, with a first coil segment 134A and a second coil segment 1348 that is adjacent the first coil segment. The components in the sheath are enlarged for illustrative purposes and not necessarily proportional to actual sizes. Sheath 120 is shown being bent at an extreme radius of curvature that approaches a limit where failure such as kinking or rupturing may begin to occur. The cross-section of the coil segments 134A, 134B is identical to coil 34D in FIG. 3E, although the cross-section of the coil can be the equivalent to any of the coil cross-sections described herein. The inner liner 150 that defines the lumen 130 and the outer layer 140 are also shown. The first coil segment 134A includes the first lateral edge 142 having curved portions 143, 145, 147, and 149 that correspond to respective curved portions 43, 45, 47, and 49 of FIG. 3E. The second coil segment 134B includes the second lateral edge 144 having the same curved portions such as curved portion 149. The inwardly curved portions 143, 149 can function as a cupped interface surface that is adapted to receive the outwardly curved portions 145, 147, as well as the material 141 therebetween that comprises the outer layer material.

The coil segments 134A, 134B are oriented such that curved portions 143, 145 are along the inner liner 150 to allow more material to be positioned along the radially outer positions in the sheath. In other words, the portion of material 141 having the enlarged distance, shown as distance 56 in FIG. 3E, is along the radial inside of the sheath 120. One benefit of this arrangement is that there can be more material along the inner liner than along the outer surface so that protrusion formation in the lumen of the sheath due from material tearing or coils sliding is further inhibited.

As shown by the arrows in FIG. 4A, there is compression along the inside surface of the curved sheath, and tension along the outside surface of the sheath 120 when in a curved configuration. Lateral edges 142, 144 of the coil segments are configured to better utilize the full compressive and/or tensile strength of the material 141. Preferably, any possible failure of the sheath due from extreme bending is not a result of kinking (compression failure along the inside of the sheath), but rather a result of rupturing (tension failure along the outside of the sheath). One advantage of transferring the failure of the sheath to rupturing, instead of kinking, is that the integrity of the luminal wall of the sheath remains intact, thereby allowing interventional devices or fluids to be safely removed.

The contour of the lateral edges of the coil cross-section may be configured to allow a more uniform compressive stress and/or tensile stress profile of the material located between adjacent lateral edges upon the bending of the sheath. The contour of the lateral edges may also be configured to reduce premature tearing of the material caused by pinching of the coil segments. In FIG. 4B is an enlarged portion of the sheath 120 in FIG. 4A depicting a compressive stress distribution, shown by the dashed lines 160, of the material 141. The compressive stress distribution 160 is a result of the material being compressed between the lateral edges 142, 144 of the respective coil segments 134A, 134B, and in particular between the outwardly curved portion 147 and the inwardly curved portion 149. As can be seen, upon bending, the curved portions 147, 149 of the adjacent coil segments move closer toward one another to compress the material 141. As a result, portions of the compressed material 141 can be displaced radially inward and outward (shown, e.g., by the bump along the outside wall of the sheath). The displaced material can form a locking notch that may mechanically obstruct the adjacent coil segments from sliding past one another. The compressive stress distribution lines indicate that, as the material 141 is compressed and displaced, it has a tendency to conform to the shape of the curved portions of the coil segments. To this end, the material 141 is essentially an extension of the shape of the outwardly curved portion 149 in order to surround the shape of the inwardly curved portion 147. A more uniform compressive stress distribution can also allow better utilization of the properties of the material so that tearing of the material from sliding coil segments is further inhibited.

FIG. 4C illustrates a sheath 120A substantially identical to the sheath 120 in FIG. 4A, except the coil segments 134A, 134B are oriented such that curved portions 143, 145 are now along outer surface, instead of the inner liner. Here, the coil cross-section is oriented to allow more material to be positioned along the radially outward positions in the sheath. In other words, the portion of material 141 having the enlarged distance, shown as distance 56 in FIG. 3E, is along the radial outside of the sheath 120A. This arrangement can provide more material along the outer surface which is the innermost surface and the outermost surface along the bent sheath. As a result, the compressive and/or tensile strength of the material 141 is better utilized along these regions in a manner to improve kink resistance and/or rupture resistance.

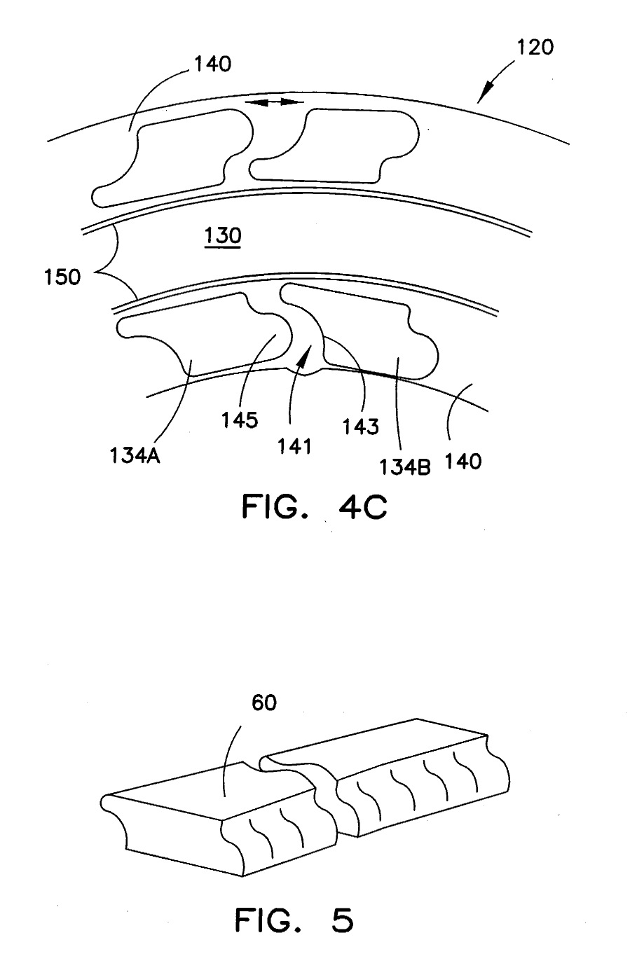

Construction of the sheath of the illustrated embodiments will be now described. The coil can go through a series of manufacturing processes in order to be desirably shaped as described herein. For example, a wire can be made through a series of drawing processes to form the desired shape as known in the art. Optionally, a wire can be micro machined by a series of process involving laser cutting and/or grinding or other processes known in the art. To better illustrate one example of the wire, FIG. 5 is a perspective view of a wire 60 before being wound as coil 34D in FIG. 3E. The wire is then wound into a coil using a lathe as known in the art.

A mandrel is selected which has a diameter at least the size of the unstressed, free inner diameter of the coil. The inner liner can be placed on the mandrel in a known matter. The coil can then be fitted or wrapped about the mandrel with the inner liner, the mandrel temporarily maintaining the coil in an expanded condition with a diameter larger than the unstressed, free inner diameter. The coil can be compression fitted or radially expanded attached during manufacture. Radially expanded fitting is described in the previously incorporated U.S. Pat. No. 6,939,337. The outer surface of the coil, as well as the outer surface of the inner liner, may be roughened for improved adhesion. When used, a braid structure can also be fitted around the coil.

The coil can be fitted by positioning a structure comprising the mandrel with the inner liner and/or the intermediate layer at the head and tail stock of a lathe. A coil transfer mechanism is mounted on the lathe carriage. The structure is rotated and the coil is wrapped thereon, as the coil transfer mechanism moves longitudinally parallel to the mandrel at the predetermined coil spacing. More details for applying a coil to form a sheath can be found in the previously incorporated U.S. Pat. No. 5,380,304. Optionally, the coil can be manually applied around the mandrel.

Finally, the outer layer, which is preferably formed from heat-shrinkable tubing, can be established over the coil. A heat reduced sleeve, e.g., FEP heat-shrinkable tubing (heat fused shrink tubing), may also be applied over the outer layer. The mandrel and the elements thereon are heated to shrink and cure the outer layer for heat setting thereof and to cause the outer layer to thermally bond to the outer surface of the coil. During heating, material of the outer layer can flow between the spacings defined by the first lateral edge of a first coil segment and the second lateral edge of a second coil segment, adjacent the first. Slight ridges may form, as the outer diameter along the spacings may be slightly smaller than the outer diameter along the coil. The mandrel and formed the sheath are then cooled, and the heat reduced sleeve is removed and the sheath is also removed from the mandrel. Additional coatings, such as hydrophilic coating and/or lubricious coating, may be applied, e.g., by spraying, dipping, brushing, additional layer and heat setting.

Those skilled in the art will appreciate that all dimensions, compositions, etc., described herein are exemplary only, and that other appropriate dimensions, compositions, etc., may be substituted in an appropriate case. For example, the respective thicknesses of the inner liner and the outer layer for a sheath are conventional, and may be varied based upon the intended use of the sheath. If desired, the sheath can be formed to have one or more segments of varying durometer along its length, typically aligned in a sequence of decreasing durometer from the proximal end to the distal end in well-known fashion. Additionally, other features commonly found in sheaths, such as radiopaque markers, rings, coatings, etc., may also be incorporated into the inventive structure in well-known manner. Although the intended use of the sheath is for medical devices, it can appreciated by those skilled in the art that the coil with the enhanced geometry described herein can be used for other applications. Furthermore, each of the cross-sections of the coils in the illustrated figures can be mirrored in the vertical or horizontal direction such that the curved portions shown need not be in the proximal or distal directions or the radial outer or inner directions as shown.

Drawings in the figures illustrating various sheath and coil embodiments are not necessarily to scale. Some drawings may have certain details magnified for emphasis, and any different numbers or proportions of parts should not be read as limiting, unless so-designated in the present disclosure. Those skilled in the art will appreciate that embodiments not expressly illustrated herein may be practiced within the scope of the present invention(s) of this disclosure, including those features described herein for different embodiments may be combined with each other and/or with currently-known or future-developed technologies while remaining within the scope of the claims presented here. It is therefore intended that the foregoing detailed description be regarded as illustrative rather than limiting. And, it should be understood that the following claims, including all equivalents, are intended to define the spirit and scope of this invention(s) of this disclosure.

1. A sheath for use in a medical device, having a proximal end and a distal end, the sheath comprising:
an inner liner defining a passageway extending longitudinally therethrough;
a coil fitted around at least a part of the inner liner, the coil having a series of windings being spaced apart longitudinally, the coil having a cross-section defined by radially inner and outer surfaces interconnected by first and second lateral edges; and
an outer layer positioned longitudinally over said coil to bond to the inner liner through spacings between adjacent coil windings,
wherein the first lateral edge of the coil comprises an inwardly curved portion and the second lateral edge of the coil comprises an outwardly curved portion.

2. The sheath of claim 1, wherein each of the inwardly curved portion of the first lateral edge of the coil and the outwardly curved portion of the second lateral edge has a radius of curvature, the inwardly curved portion radius being at least as large as the outwardly curved portion radius.

3. The sheath of claim 1, wherein the first lateral edge of the coil further comprises an outwardly curved portion joined to the inwardly curved portion.

4. The sheath of claim 3, wherein each of the inwardly curved portion of the first lateral edge of the coil and the outwardly curved portion of the first lateral edge has a radius of curvature, the inwardly curved portion radius of the first lateral edge being at least as large as the outwardly curved portion radius of the first lateral edge.

5. The sheath of claim 3, wherein the coil cross-section further comprises an outer portion disposed along the outer layer and an inner portion disposed along the inner liner, where at least one of the outer portion laterally extends past the inner portion and the inner portion laterally extends past the outer portion.

6. The sheath of claim 1, wherein the second lateral edge of the coil further comprises an inwardly curved portion joined to the outwardly curved portion.

7. The sheath of claim 6, wherein each of the inwardly curved portion of the second lateral edge of the coil and the outwardly curved portion of the second lateral edge has a radius of curvature, the outwardly curved portion radius of the second lateral edge being at least as large as the inwardly curved portion radius of the second lateral edge.

8. The sheath of claim 1, wherein the first lateral edge of the coil further comprises an outwardly curved portion joined to the inwardly curved portion, and the second lateral edge of the coil further comprises an inwardly curved portion joined to the outwardly curved portion.

9. The sheath of claim 8, wherein each of the outwardly curved portion of the first lateral edge of the coil and the inwardly curved portion of the second lateral edge has a radius of curvature, the inwardly curved portion radius of the second lateral edge being at least as large as the outwardly curved portion radius of the first lateral edge.

10. The sheath of claim 8, wherein each of the curved portions has a radius of curvature, where at least one of:
the inwardly curved portion radius of the first lateral edge being at least as large as the inwardly curved portion radius of the second lateral edge, and
the outwardly curved portion radius of the second lateral edge at least as large at the outwardly curved portion radius of the first lateral edge.

11. The sheath of claim 8, wherein the coil cross-section further comprises an outer portion disposed along the outer layer and an inner portion disposed along the inner liner, where at least one of the outer portion and the inner portion laterally extends past the respective inner and outer portions.

12. The sheath of claim 11, wherein the laterally extended portion of the at least one of outer portion and the inner portion defines said outwardly curved portion.

13. The sheath of claim 1, where the coil cross-section further comprises an outer portion disposed along the outer layer and an inner portion disposed along the inner liner, where the first lateral edge of a coil segment and the second lateral edge of an adjacent coil segment are structured and arranged so that a lateral distance between the coil segments is different between the outer portion than the inner portion.

14. A sheath for use in medical devices, having a proximal end and a distal end, the sheath comprising:
an inner liner defining a passageway extending longitudinally therethrough;
a coil fitted around at least a part of the inner liner, the coil having a series of windings being spaced apart longitudinally, the coil having a cross-section defined by radially inner and outer surfaces interconnected by first and second lateral edges; and
an outer layer positioned longitudinally over said coil to bond to the inner liner through spacings between adjacent coil windings,
wherein the first lateral edge of a coil winding segment of said coil and the second lateral edge of an adjacent coil winding segment of said coil are structured and arranged to define an asymmetric spacing therebetween, said asymmetric spacing configured to improve at least one of kink resistance and rupture resistance of the sheath when in a bent configuration.

15. The sheath of claim 14, wherein along one of the first and second lateral edges a portion is inwardly curved at a radius of curvature, and along the other of the first and second lateral edges a portion is outwardly curved at a radius of curvature, where the inwardly curved radius of the one of the first and second lateral edges is at least as large as the outwardly curved radius of the other of the first and second lateral edges.

16. The sheath of claim 15, wherein the cross-section includes an outer portion disposed along the outer layer and an inner portion disposed along the inner liner, at least one of the outer portion laterally extends past the inner portion along a first of the first and second lateral edges and the inner portion laterally extends past the outer portion along a second of the first and second lateral edges.

17. The sheath of claim 16, wherein the outer portions of the coil winding segment and the adjacent coil winding segment are spaced at a first distance, the inner portions of the coil winding segment and the adjacent coil winding segment are spaced at a second distance, where the first distance is greater than the second distance.

18. The sheath of claim 16, wherein the outer portions of the coil winding segment and the adjacent coil winding segment are spaced at a first distance, the inner portions of the coil winding segment and the adjacent coil winding segment are spaced at a second distance, where the second distance is greater than the first distance.

19. The sheath of claim 16, wherein the inwardly curved portion of the one of the first and second lateral edges of the coil winding segment and the outwardly curved portion of the other of the first and second lateral edges of the adjacent coil winding segment are structured and arranged with outer layer material therebetween to form a ball-and-socket interface.

20. A method of forming a sheath, comprising:
providing an inner polymer liner, said inner liner having a passageway extending therethrough and an outer surface;
positioning said polymer inner liner around a mandrel;
positioning a coil around the inner polymer liner, said coil having a series of windings being spaced apart longitudinally, the coil being further defined by a cross-section comprising radially inner and outer surfaces interconnected by first and second lateral edges, wherein the first lateral edge of the coil comprises an inwardly curved portion and the second lateral edge of the coil comprises an outwardly curved portion;
applying an outer polymer layer over at least a portion of said coil; and
exposing an assembly comprising the mandrel, inner polymer liner, coil, and outer polymer layer to a sufficient amount of heat to at least partially melt the outer polymer layer such that a bond is formed between outer polymer layer and the inner polymer liner.
